(19) United States
(12) Patent Application Publication
(10) Pub. No.: US 2003/0186316 A1
(43) Pub. Date: Oct. 2, 2003

(54) Method for identifying optimal binding ligands to a receptor

(75) Inventors: William D. Huse, Del Mar, CA (US); Michael H. Freedman, Del Mar, CA (US)

Correspondence Address: CAMPBELL & FLORES LLP, 4370 LA JOLLA VILLAGE DRIVE, 7TH FLOOR, SAN DIEGO, CA, 92122, US

(73) Assignee: IXSYS, INCORPORATED.

(21) Appl. No.: 09/839,469

(22) Filed: Apr. 20, 2001

Related U.S. Application Data
(62) Division of application No. 09/169,048, filed on Oct. 8, 1998.
(60) Provisional application No. 60/112,011, filed on Oct. 9, 1997.

(51) Int. Cl. G01N 33/53

(52) U.S. Cl. 435/7.1

(57) ABSTRACT

The present invention provides a method for determining binding of a receptor to one or more ligands. The method consists of contacting a collective receptor variant population with one or more ligands and detecting binding of one or more ligands to the collective receptor variant population. The collective receptor variant population can be further divided into two or more subpopulations, one or more of the two or more subpopulations can be contacted with one or more ligands and one or more receptor variant subpopulations having binding activity to one or more ligands can be detected. The steps of dividing, contacting and detecting can be repeated one or more times. The invention also provides methods for identifying a receptor variant having optimal binding activity to one or more ligands. The invention additionally provides a method for determining binding of a ligand to one or more receptors. The method consists of contacting a collective ligand variant population with one or more receptors and detecting binding of one or more receptors to the collective ligand variant population. As with the variant receptor population, the methods for determining binding of a ligand to one or more receptors can include the steps of further dividing, contacting and detecting one or more ligand variants having binding activity to one or more receptors. The invention also provides methods for identifying a ligand or ligand variant having optimal binding activity.

This application claims the benefit of priority of U.S. Ser. No. 08/948,187, filed Oct. 9, 1997, which was converted to a U.S. Provisional Application, the entire contents of which is incorporated herein by reference.

### BACKGROUND OF THE INVENTION

The present invention relates generally to receptor-ligand binding interactions and more specifically to methods for determining the optimal binding partner for a ligand or receptor.

The development of new and more effective drugs is a primary goal of the pharmaceutical industry. Drug discovery and development can be described as following two general approaches, screening for lead compounds and structure-based drug design.

Drug discovery based on screening for lead compounds involves generating a pool of candidate compounds. These candidate compounds can be derived from natural products, such as plants, insects or other organisms. The pool of candidate compounds can also be recombinantly generated such as with phage display libraries of combinatorial antibody libraries and random peptide libraries. Alternatively, the candidate compounds can be chemically synthesized using approaches such as combinatorial chemistry in which compounds are synthesized by combining chemical groups to generate a large number of diverse candidate compounds.

Generally, the pool of candidate compounds is screened with a drug target of interest to identify potential lead compounds. This approach usually requires assaying large numbers of compounds for a desired activity. Depending on the assay, compound availability and preparation, the screening of a pool of candidate compounds can be laborious and time consuming. Moreover, further rounds of manipulations such as the screening of modified forms of the lead compound are additionally performed to determine a structure with optimal activity. Thus, these additional manipulations further complicate and increase the time and labor required for the development of a drug candidate which exhibits optimal binding activity to the target of interest.

Drug discovery and development relying on structure-based drug design uses a three-dimensional structure prediction of the drug target as a template to model compounds which inhibit or otherwise interfere with critical residues that are required for activity in the target molecule. Model compounds which show activity toward the drug target are then used as lead compounds for the development of candidate drugs which exhibit a desired activity toward the drug target.

Identifying model compounds using structure-based drug design can provide advantages in predicting modifications of the lead compound that will likely improve binding of the compound to the drug target. However, obtaining structures of relevant drug targets is extremely time consuming and laborious. Moreover, successive rounds of modifications and testing to identify a compound which exhibits a desired binding activity toward the drug target is similarly laborious and time consuming. Such a process often takes years to accomplish. In addition, if the drug target of interest is a receptor on the surface of cells, it can be embedded in the cell membrane. Determination of the three-dimensional structures of such membrane proteins is extremely difficult as evidenced by the limited number of membrane protein structures currently available.

Thus, there exists a need for rapid and efficient methods to identify ligands that exhibit optimal binding activity to a receptor. The present invention satisfies this need and provides related advantages as well.

### SUMMARY OF THE INVENTION

The present invention provides a method for determining binding of a receptor to one or more ligands. The method consists of contacting a collective receptor variant population with one or more ligands and detecting binding of one or more ligands to the collective receptor variant population. The collective receptor variant population can be further divided into two or more subpopulations, one or more of the two or more subpopulations can be contacted with one or more ligands and one or more receptor variant subpopulations having binding activity to one or more ligands can be detected. The steps of dividing, contacting and detecting can be repeated one or more times. The invention also provides methods for identifying a receptor variant having optimal binding activity to one or more ligands. The invention additionally provides a method for determining binding of a ligand to one or more receptors. The method consists of contacting a collective ligand variant population with one or more receptors and detecting binding of one or more receptors to the collective ligand variant population. As with the variant receptor population, the methods for determining binding of a ligand to one or more receptors can include the steps of further dividing, contacting and detecting one or more ligand variants having binding activity to one or more receptors. The invention also provides methods for identifying a ligand or ligand variant having optimal binding activity.

### BRIEF DESCRIPTION OF THE DRAWINGS

FIG. 1 shows binding of chemical ligand, represented as a point in space designated X, to a receptor, represented as a disc. The bottom panel shows distribution of ligands where open circles represent diverse ligands and closed circles represent focused ligands.

FIG. 2 shows identification of an optimal binding ligand using a receptor represented as three discs and a ligand represented as three points designated X.

FIG. 3 shows binding of anti-idiotypic antibody ligands to BR96 antibody receptor variants.

FIG. 4 shows identification of an optimal binding anti-idiotypic antibody ligand that binds to multiple antibody receptor variants.

### DETAILED DESCRIPTION OF THE INVENTION

The invention provides rapid and efficient methods for determining optimal ligand-receptor binding partners. The methods are applicable for the identification of specific ligands to desired target molecules. Such ligands can be developed as potential drug candidates or, alternatively, used as lead compounds for the generation and identification of ligand variants which exhibit enhanced activity of the desired binding property. The methods are advantageous in that they use a population of receptor variants to rapidly identify ligands that have a high likelihood of binding to the target receptor molecule. By initially screening with a population of variants to the target receptor, the probability of detecting binding events is increased. Obtaining increased binding events is productive because the use of receptor variants that are all related to a parent receptor results in the identification of binding events similar to the parent receptor and, therefore, ligands identified by such a screen are similarly related to those ligands that will associate with and bind to the parent receptor. Therefore, the initial screen using a population of variants results in the rapid identification and enrichment for ligands having favorable binding characteristics toward the target receptor. This enriched population can then be subsequently screened for ligands having optimal binding characteristics toward the target receptor. The methods of the invention therefore provide a rapid and efficient method for the identification of specific ligands which are applicable for the diagnosis and treatment of diseases.

As used herein, the term “receptor” is intended to refer to a molecule of sufficient size so as to be capable of selectively binding a ligand. Such molecules generally are macromolecules, such as polypeptides, nucleic acids, carbohydrate or lipid. However, derivatives, analogues and mimetic compounds as well as natural or synthetic organic compounds are also intended to be included within the definition of this term. The size of a receptor is not important so long as the receptor exhibits or can be made to exhibit selective binding activity to a ligand. Furthermore, the receptor can be a fragment or modified form of the entire molecule so long as it exhibits selective binding to a desired ligand. For example, if the receptor is a polypeptide, a fragment or domain of the native polypeptide which maintains substantially the same binding selectivity as the intact polypeptide is intended to be included within the definition of the term receptor. Specific examples of such a binding domain or fragment is the variable region of an antibody molecule. Complementarity determining regions (CDR) within the variable region can also exhibit substantially the same binding selectivity as the antibody molecule and are therefore considered to be within the meaning of the term.

In one embodiment, an optimal binding ligand is identified by generating a population of G protein coupled receptor variants. The G protein coupled receptor variants are pooled into a collective receptor variant population and screened for binding activity to ligands within a diverse population. The receptor variant population can be screened by dividing the ligand population into subpopulations or individual ligands to determine binding activity. The binding activity of ligands exhibiting binding to the receptor variant population are compared to identify a ligand having optimal binding characteristics. More preferred binding ligands can be subsequently identified by generating a library of ligand variants based on the identified optimal binding ligand and screening for binding activity to the parent G protein coupled receptor. The binding activity of positive binding ligand variants are compared to each other and to the parent ligand to identify the ligand or ligands which exhibits preferred or optimal binding characteristics to the parent receptor.

Receptors can include, for example, cell surface receptors such as G protein coupled receptors, integrins, growth factor receptors and cytokine receptors. In addition to antibodies, receptors can include other polypeptides or ligands of the immune system. Such other polypeptides of the immune system include, for example, T cell receptors (TCR), major histocompatibility complex (MHC), CD4 receptor and CD8 receptor. Furthermore, cytoplasmic receptors such as steroid hormone receptors and DNA binding polypeptides such as transcription factors and DNA replication factors are likewise included within the definition of the term receptor.

As used herein, the term “polypeptide” when used in reference to a receptor or a ligand is intended to refer to peptide, polypeptide or protein of two or more amino acids. The term is similarly intended to refer to derivatives, analogues and functional mimetics thereof. For example, derivatives can include chemical modifications of the polypeptide such as alkylation, acylation, carbamylation, iodination, or any modification which derivatizes the polypeptide. Analogues can include modified amino acids, for example, hydroxyproline or carboxyglutamate, and can include amino acids that are not linked by peptide bonds. Mimetics encompass chemicals containing chemical moieties that mimic the function of the polypeptide regardless of the predicted three-dimensional structure of the compound. For example, if a polypeptide contains two charged chemical moieties in a functional domain, a mimetic places two charged chemical moieties in a spatial orientation and constrained structure so that the charged chemical function is maintained in three-dimensional space. Thus, all of these modifications are included within the term “polypeptide” so long as the polypeptide retains its binding function.

As used herein, the term “ligand” refers to a molecule that can selectively bind to a receptor. The term selectively means that the binding interaction is detectable over non-specific interactions by a quantifiable assay. A ligand can be essentially any type of molecule such as polypeptide, nucleic acid, carbohydrate, lipid, or any organic derived compound. Moreover, derivatives, analogues and mimetic compounds are also intended-to be included within the definition of this term. As such, a molecule that is a ligand can also be a receptor and, conversely, a molecule that is a receptor can also be a ligand since ligands and receptors are defined as binding partners. Those skilled in the art know what is intended by the meaning of the term ligand. Specific examples of ligands are natural or synthetic organic compounds as well as recombinantly or synthetically produced polypeptides. Such polypeptides that bind to receptor variants are described below in Example V.

As used herein, the term “variant” when used in reference to a receptor or ligand is intended to refer to a molecule that shares a similar structure and function. The characteristics that define the function can be determined by a parent receptor or by a parent ligand. Variants possess, for example, substantially the same or similar binding function as the parent molecule. However, variants can have a detectable difference in the chemical functional groups of the binding function and still be considered a variant of the parent molecule. Variants include, for example, parent receptors that are directly modified such as by the mutation of an amino acid residue or the addition of a chemical moiety. Modifications can also be indirect such as the binding of a regulatory molecule or allosteric effector which alters the binding function of the parent receptor.

Additionally, the variant can be an isoform or family member that is distinct but related to the parent receptor. All of such direct or indirect modifications of a parent molecule as well as related members thereof are considered to be within the definition of the term variant as used herein. Chemical functional groups that differ from the parent molecule can be used to generate a population of variant molecules. In the specific example of a polypeptide receptor parent, a variant can differ by, for example, one or more amino acids in a functional binding domain. In this specific example, a functional binding domain refers to a region or a portion of the polypeptide that contributes to binding interactions between the receptor and ligand. Such functional binding domains include, for example, both catalytic domains and ligand binding domains, as well as structural domains that contribute to the polypeptide function.

As used herein, the term “population” is intended to refer to a group of two or more different molecules. A population can be as large as the number of individual molecules currently available to the user or able to be made by one skilled in the art. Typically, populations can be as small as 2 molecules and as large as 10¹³ molecules. In some embodiments, populations are between about 5 and 10 different species as well as up to hundreds or thousands of different species. In the specific example presented in Example V, the population described therein is 7 different species. In other embodiments, populations can be, for example, greater than 10⁵, 10⁶ and 10⁸ different species. In yet other embodiments, populations are between about 10⁸-10¹² or more different species. Moreover, the populations can be diverse or redundant depending on the intent and needs of the user. Those skilled in the art will know what size and diversity of a population is suitable for a particular application.

As used herein, the term “subpopulation” refers to a subgroup of one or more species of molecules from an original population. The subpopulation can be obtained by, for example, dividing the population into one or more fractions or synthesizing or generating a known fraction of the original population. The subpopulation need not contain equivalent numbers of different molecules.

As used herein, the term “collective,” when used in reference to populations or subpopulations, refers to an aggregate of members that form the population or subpopulation.

As used herein, the term “optimal binding” refers to a preferred binding characteristic of a ligand and receptor interaction. Optimal binding can be ligand-receptor interactions of a desired affinity, avidity or specificity. For example, optimal binding can be interactions that are most effective in a biological assay. The optimal binding characteristics will depend on the particular application of the binding molecule. For example, the binding standard can be relative affinity of a ligand for the parent receptor. In this case, a ligand in a population with the highest binding affinity to a parent receptor would have optimal binding. Alternatively, the standard can be the highest binding affinity of a ligand subpopulation to a receptor variant subpopulation. In this example, the ligand subpopulation with highest affinity for a receptor variant subpopulation would have optimal binding. In this case, the highest affinity ligand would be a member of the ligand subpopulation and, likewise, the highest affinity receptor variant would be a member of the receptor variant subpopulation. Optimal binding also can be binding to the largest number of receptor variants or binding to greater than some threshold number of receptor variants.

The invention provides a method for determining binding of a receptor to one or more ligands by contacting a collective receptor variant population with one or more ligands and detecting binding of one or more ligands to the collective receptor variant population.

The methods of the invention employ a collective population of variant but similar molecules to screen one or more binding partners for a detectable interaction. For example, a collective receptor variant population is screened with one or more ligands to determine binding activity. Using a receptor variant population is advantageous in that the receptor variant population provides an expanded receptor target range compared to a single receptor of similar function for the identification of binding ligands. This expanded target range increases the probability that at least one ligand in a population will have detectable binding affinity for a receptor variant.

Increased probability of detecting binding ligands to a population of variant receptors has practical applications in that a large number of different ligands can be screened with a single variant population to rapidly identify a subset of the ligand population that is most likely to have desired binding properties toward the preferred or parent receptor. Essentially, the use of a population of variant receptors to identify binding partners eliminates in an initial screen ligands that are unlikely to bind the parent receptor. The subpopulation of ligands that exhibit binding to the variant receptor population can be subsequently tested for binding activity and affinity toward the parent receptor. Moreover, if the initial subpopulation of ligands remains relatively large, further screens using subpopulations of variant receptors that reduce the receptor target binding range to variants more closely related to the parent receptor can be performed to narrow the likely binding ligands that exhibit preferential binding characteristics.

In addition to rapidly identifying binding ligands that have a high probability of binding to a desired receptor, the use of an expanded binding target range similarly allows for the rapid identification of a receptor that binds to a particular ligand. In this case, a population of receptors can be screened with a ligand variant population in similar fashion to that described above in which the receptors which are unlikely to bind to the parent ligand are eliminated. Similarly, the ligand binding range can be reduced by subsequently using ligand variants that are more closely related to the parent ligand so as to preferentially identify receptors that exhibit desired binding characteristics.

Screening variant populations of receptors or ligands to rapidly identify likely binding partners has the added advantage that such a screen will also identify a greater range of binding candidates, including binding partners that exhibit low or undetectable binding toward the parent molecule. For example, the increased probability of detecting a ligand interaction with a receptor variant population can be exemplified in the context of complementary interactions between receptors and ligands. For example, the affinity of a ligand for a receptor can be determined by the chemical functional groups at the site of contact between the receptor and ligand and the relative position of the chemical groups in three-dimensional space. Receptor variants and ligand variants can, for example, differ in chemical functional groups in their contact sites or differ in other chemical functional groups that contribute to the conformation and three-dimensional orientation of the chemical functional groups in the contact site. A receptor variant population contains receptor variants that can differ in the ligand contact site or sites and therefore can have different affinities for different ligands. A ligand can have an affinity for the parent receptor below the level of detectable binding. In contrast, the same ligand can exhibit detectable and even strong binding affinity for a receptor variant. Screening the ligand against the parent receptor would not allow the identification of the ligand as a binding partner. Using a receptor variant population therefore increases the likelihood of identifying ligands that bind to the parent receptor regardless of affinity. Having the capability of identifying ligands independent of its binding strength allows the selection of a ligand exhibiting a relative affinity suitable for an intended purpose.

In addition, screening with a receptor variant population provides additional information about the relative affinity of a given binding ligand for a target receptor. For example, a ligand that binds to a larger number of receptor variants has an increased likelihood of binding to the target or parent receptor than one that binds to fewer receptor variants such as only one receptor variant. Thus, more information is obtained when ligands are screened with a receptor variant population than when ligands are screened with the parent receptor alone.

Additionally, the binding ligands identified using methods of the invention can be used to generate a library of ligand variants. The identified ligand is used as a parent ligand to generate a library containing a ligand variant population. The library of ligand variants can be based on structural similarities to the parent ligand, for example, such libraries of ligand variants can be generated using combinatorial chemistry methods (Combinatorial Peptide and Nonpeptide Libraries: A Handbook, Jung, ed., VCH, New York (1996)).

The characteristics of the receptor variants can be varied depending on the needs of a particular ligand screen. For example, if the receptor variants are closely related, then a ligand that binds to the most number of receptor variants has the greatest likelihood of binding to the parent receptor. The characteristics of the receptor variants can also be varied so that the receptor variants in a population are less closely related. Thus, depending on the needs of the investigator, the receptor variants can be made to be more or less closely related.

The relatedness of the receptor variant to the parent receptor can be determined by the chemical similarities or differences of the particular chemical functional groups that define the receptor variant relative to the analogous chemical functional group in the parent receptor. For example, if the parent receptor or ligand is a polypeptide, the relatedness of the variants to the parent is determined by the relatedness of the amino acids that differ between the variants and the parent molecule. A chemically more conservative difference between the variant and the parent results in variants more closely related to the parent molecule. Conservative substitutions of amino acids include, for example, (1) non-polar amino acids (Gly, Ala, Val, Leu and Ile); (2) polar neutral amino acids (Cys, Met, Ser, Thr, Asn and Gln); (3) polar acidic amino acids (Asp and Glu); (4) polar basic amino acids (Lys, Arg and His); and (5) aromatic amino acids (Phe, Tyr, Trp and His). Additionally, conservative substitutions of amino acids include, for example, substitutions based on the frequencies of amino acid changes between corresponding proteins of homologous organisms (Principles of Protein Structure, Schulz and Schirmer, eds., Springer Verlag, New York (1979)).

A ligand generally interacts with a receptor through multiple molecular interactions resulting from multiple contact points or through multiple interactions of a chemical functional group that can be described, for example, as three points. These three points can be, for example, three distinct chemical groups that serve as contact points for the binding partner. Likewise, three different amino acids or three different clusters of amino acids in a polypeptide ligand or receptor can serve as contact points for the binding partner. In this case, binding between the ligand and receptor will occur only when all three points can bind.

Using the above multiple-point binding description for ligand-receptor interactions, a receptor variant population can be generated in which one of the points is fixed so that it is identical to the parent receptor and the other points are varied to generate a receptor variant population. For example, using three reference points, one point is fixed to be identical to the parent receptor and the other two points are varied to generate a receptor variant population. By generating a receptor variant population, the probability of detecting binding of a ligand to one of the receptor variants is increased. Identification of a binding ligand can then be performed as an iterative process. A ligand identified by fixing one point and varying the other contact points on the receptor can be used to generate a library of ligand variants. In the next iteration of the process, the original receptor contact point can be fixed and an additional point can be fixed to be identical to the parent receptor. In the example above describing three reference points, two points are fixed to be identical to the parent receptor and one point is varied to generate a second receptor variant population. The library of ligand variants is screened with the second receptor variant population to identify binding ligands from the ligand variant library. The binding activity of the identified binding ligands can be compared to identify a ligand variant having optimal binding activity to the parent receptor. The process of fixing additional receptor contact points, identifying one or more ligand variants with optimal binding and generating a library of ligand variants is repeated until a ligand is identified that binds to the parent receptor with optimal activity. Thus, a population of ligands or a population of ligand variants can be screened with different receptor variant populations derived from the same parent receptor to identify binding ligands.

A parent receptor can be any molecule that binds to a ligand. The receptors can be, for example, cell surface receptors that transmit intracellular signals upon binding of a ligand. For example, the G protein coupled receptors span the membrane seven times and couple signaling to intracellular heterotrimeric G proteins. G protein coupled receptors participate in a wide range of physiological functions, including hormonal signaling, vision, taste and olfaction. Moreover, these receptors encompass a large family of receptors, including receptors for acetylcholine, adenosine and adenine nucleotides, β-adrenergic ligands such as epinephrine, angiotensin, bombesin, bradykinin, cannabinoids, chemokines, dopamine, endothelin, histamine, melanocortins, melanotonin, neuropeptide Y, neurotensin, opioid peptides, platelet activating factor, prostanoids, serotonin, somatostatin, tachykinin, thrombin and vasopressin, among others.

Other cell surface receptors have intrinsic tyrosine kinase activity and include growth factor or hormone receptors for ligands such as platelet-derived growth factor, epidermal growth factor, insulin, insulin-like growth factor, hepatocyte growth factor, and other growth factors and hormones. In addition, cell surface receptors that couple to intracellular tyrosine kinases include cytokine receptors such as those for the interleukins and interferons.

Integrins are cell surface receptors involved in a variety of physiological processes such as cell attachment, cell migration and cell proliferation. Integrins mediate both cell-cell and cell-extracellular matrix adhesion events. Structurally, integrins consist of heterodimeric polypeptides where α single a chain polypeptide noncovalently associates with a single β chain. In general, different binding specificities are derived from unique combinations of distinct α and β chain polypeptides. For example, vitronectin binding integrins contain the αᵥ integrin subunit and include αᵥβ₃, αᵥβ₁ and α_{v β}₅, all of which exhibit different ligand binding specificities.

Receptors also can function in the immune system. An antibody or immunoglobulin is an immune system receptor which binds to a ligand. The polypeptide receptor can be the entire antibody or it can be any functional fragment thereof which binds to the ligand. Functional fragments such as Fab, F(ab)₂, Fv, single chain Fv (scFv) and the like are included within the definition of the term antibody. The use of these terms in describing functional fragments of an antibody are intended to correspond to the definitions well known to those skilled in the art. Such terms are described in, for example, Harlow and Lane, Antibodies: A Laboratory Manual, Cold Spring Harbor Laboratory, New York (1989), which is incorporated herein by reference.

As with the above terms used for describing antibodies and functional fragments thereof, the use of terms which reference other antibody domains, functional fragments, regions, nucleotide and amino acid sequences and polypeptides or peptides, is similarly intended to fall within the scope of the meaning of each term as it is known and used within the art. Such terms include, for example, “heavy chain polypeptide” or “heavy chain”, “light chain polypeptide” or “light chain”, “heavy chain variable region” (V_{H}) and “light chain variable region” (V_{L}) as well as the term “complementarity determining region” (CDR).

In addition to antibodies, the receptors can be T cell receptors (TCR). T cell receptors contain two subunits, α and β, which are similar to antibody variable region sequences in both structure and function. In this regard, both subunits contain variable region which encode CDR regions similar to those found in antibodies (Immunology, Third Ed., Kuby, J. (ed.), New York, W. H. Freeman & Co. (1997)). The CDR containing variable regions of TCRs bind to antigens presented on the cell surface of antigen-presenting cells and are capable of exhibiting binding specificities to essentially any particular antigen.

Other exemplary receptors of the immune system which exhibit known or inherent binding functions include major histocompatiblility complex (MHC), CD4 and CD8. MHC functions in mediating interactions between antigen-presenting cells and effector T cells. CD4 and CD8 receptors function in binding interactions between effector T cells and antigen-presenting cells. CD4 and CD8 also exhibit similar CDR region structure as do antibodies and TCRs sequences.

The generation of receptor variant populations can be by any means desired by the user. Those skilled in the art will know what methods can be used to generate receptor variants. For example, receptor variants of a given polypeptide receptor can be generated by mutagenesis of one or more amino acids in functional domains so long as the receptor variant retains a structural or functional similarity to the parent receptor. In such a case, mutagenesis of the receptor can be carried out using methods well known to those skilled in the art (Molecular Cloning: A Laboratory Manual, Sambrook et al., eds., Cold Spring Harbor Press, Plainview, N.Y. (1989)). For example, in the case of G protein coupled receptors, the extracellular domain can be identified based on sequence homology and topology of the seven membrane spanning domains of this class of receptors. Mutagenesis of the regions corresponding to the extracellular domain can provide a receptor variant population useful for screening ligands that bind to and elicit a signaling response from the parent G protein coupled receptor.

One method well known in the art for rapidly and efficiently producing a large number of alterations in a known amino acid sequence or for generating a diverse population of random sequences is known as codon-based synthesis or mutagenesis. This method is the subject matter of U.S. Pat. Nos. 5,264,563 and 5,523,388 and is also described in Glaser et al. J. Immunology 149:3903-3913 (1992). Briefly, coupling reactions for the randomization of, for example, all twenty codons which specify the amino acids of the genetic code are performed in separate reaction vessels and randomization for a particular codon position occurs by mixing the products of each of the reaction vessels. Following mixing, the randomized reaction products corresponding to codons encoding an equal mixture of all twenty amino acids are then divided into separate reaction vessels for the synthesis of each randomized codon at the next position. For the synthesis of equal frequencies of all twenty amino acids, up to two codons can be synthesized in each reaction vessel.

Variations to these synthesis methods also exist and include for example, the synthesis of predetermined codons at desired positions and the biased synthesis of a predetermined sequence at one or more codon positions. Biased synthesis involves the use of two reaction vessels where the predetermined or parent codon is synthesized in one vessel and the random codon sequence is synthesized in the second vessel. The second vessel can be divided into multiple reaction vessels such as that described above for the synthesis of codons specifying totally random amino acids at a particular position. Alternatively, a population of degenerate codons can be synthesized in the second reaction vessel such as through the coupling of XXG/T nucleotides where X is a mixture of all four nucleotides. Following synthesis of the predetermined and random codons, the reaction products in each of the two reaction vessels are mixed and then redivided into an additional two vessels for synthesis at the next codon position.

A modification to the above-described codon-based synthesis for producing a diverse number of variant sequences can similarly be employed for the production of the variant populations described herein. This modification is based on the two vessel method described above which biases synthesis toward the parent sequence and allows the user to separate the variants into populations containing a specified number of codon ositions that have random codon changes.

Briefly, this synthesis is performed by continuing to divide the reaction vessels after the synthesis of each codon position into two new vessels. After the division, the reaction products from each consecutive pair of reaction vessels, starting with the second vessel, is mixed. This mixing brings together the reaction products having the same number of codon positions with random changes. Synthesis proceeds by then dividing the products of the first and last vessel and the newly mixed products from each consecutive pair of reaction vessels and redividing into two new vessels. In one of the new vessels, the parent codon is synthesized and in the second vessel, the random codon is synthesized. For example, synthesis at the first codon position entails synthesis of the parent codon in one reaction vessel and synthesis of a random codon in the second reaction vessel. For. synthesis at the second codon position, each of the first two reaction vessels is divided into two vessels yielding two pairs of vessels. For each pair, a parent codon is synthesized in one of the vessels and a random codon is synthesized in the second vessel. When arranged linearly, the reaction products in the second and third vessels are mixed to bring together those products having random codon sequences at single codon positions. This mixing also reduces the product populations to three, which are the starting populations for the next round of synthesis. Similarly, for the third, fourth and each remaining position, each reaction product population for the preceding position are divided and a parent and random codon synthesized.

Following the above modification of codon-based synthesis, populations containing random codon changes at one, two, three and four positions as well as others can be conveniently separated out and used based on the need of the individual. Moreover, this synthesis scheme also allows enrichment of the populations for the randomized sequences over the parent sequence since the vessel containing only the parent sequence synthesis is similarly separated out from the random codon synthesis.

Populations of receptor variants can be alternatively derived from a family of related receptors. Again using G protein coupled receptors as an example, a receptor variant population can be a collection of G protein coupled receptor family members. Because these proteins are structurally similar and carry out similar functions, they constitute a family of structurally related receptor variants that function in ligand binding. Such a receptor family can be isolated using available sequence information on the receptors and generating primers that can amplify the receptor family or generating probes that can be used to isolate genes of the family members.

In addition, a population of receptor variants can be generated from a family of related receptors even when all members of the family have not been identified. In this case, a receptor of interest is identified and related family members are isolated by, for example, generating probes that allow isolation of the related family members or by generating primers that hybridize with conserved structural domains of the parent receptor and amplifying related family members.

Once a receptor has been identified and a variant receptor population has been generated, the receptor variants are produced in a manner convenient for detecting ligand binding to a collective receptor variant population. One such system involves expressing receptor variants in cells such that binding of ligands to the receptor variants can be detected in culture. One detection method is based on utilizing the cellular signaling properties of the receptor to detect binding of a ligand. Utilizing the signaling properties of the receptor variants is convenient because it allows detection of ligand binding without the need to isolate and purify the receptor variant population or to prepare cell extracts for in vitro assays.

One system for detecting cellular signaling events is the melanophore system (Lerner, Trends Neurosci. 17:142-146 (1994)). Melanophores are skin cells that provide pigmentation to an organism. The equivalent cells in humans are melanocytes, which are responsible for skin and hair color. In numerous animals, including fish, lizards and amphibians, melanophores are used, for example, for camouflage. The color of the melanophore is dependent on the intracellular position of melanin-containing organelles, called melanosomes. Melanosomes move along a microtubule network and are clustered to give a light color or dispersed to give a dark color. The distribution of melanosomes is regulated by G protein coupled receptors and cellular signaling events, where increased concentrations of second messengers such as cyclic AMP and diacylglycerol results in melanosome dispersion and darkening of the melanophores. Conversely, decreased concentrations of cyclic AMP and diacylglycerol results in melanosome aggregation and lightening of the melanophores.

The level of second messengers is regulated by hormones. Melatonin stimulates receptors that lower intracellular second messenger levels and thus causes the cells to lighten. In contrast, melanocyte stimulating hormone (MSH) increases intracellular second messenger levels and causes the melanophores to darken. Other regulators of melanosome distribution include catecholamines, endothelins and light. Thus, cells darken in response to photostimulation.

The melanophore system is advantageous for testing receptor-ligand interactions including G protein coupled receptors due to the regulation of melanosome distribution by receptor stimulated intracellular signaling. For example, a G protein coupled receptor can be selected as the parent receptor and a receptor variant population can be generated. The receptor variant population is transfected into melanophore cells, for example, frog melanophore cells, and the G protein coupled receptor variants are expressed. Ligands that stimulate or inhibit G protein coupled receptor signaling can be determined since the system can be used to detect both aggregation of melanosomes and lightening of cells and dispersion of melanosomes and darkening of cells.

In addition to G protein coupled receptors, the melanophore system is also useful for testing other types of receptors so long as the receptors couple into a signaling mechanism that regulates melanosome distribution. For example, many receptor tyrosine kinases couple to changes in diacylglycerol. Since diacylglycerol is a second messenger that regulates melanosome distribution, ligands that function as agonists or antagonists of these receptors or that stimulate or inhibit their tyrosine kinase activity can be analyzed using the melanophore system.

In addition to the melanophore system, other systems can be used to detect signaling events of receptors. Receptors often initiate intracellular signaling events that induce the expression of early response genes. For example, many receptor tyrosine kinases induce the early response gene fos. A reporter system can be generated, for example, by fusing the fos promoter to a detectable protein such as luciferase. Ligands that stimulate or inhibit cellular signaling from these receptors can be detected using the endogenous cellular signaling machinery without the need to perform time consuming in vitro assays.

A collective receptor variant population is contacted with one or more ligands by incubating the ligands under conditions that allow binding. For example, the ligands can be contacted and incubated with the collective receptor variant population under conditions similar to physiological conditions, such as incubation in isotonic solution at 37° C. Unbound ligands are removed from the collective receptor variant population and binding of ligands to receptor variants is detected. For example, the darkening or lightening of melanophore cells can be used to detect binding of a ligand to a receptor variant.

The invention provides methods for contacting a collective receptor variant population with one or more ligands and detecting ligand binding to the collective receptor variant population. An additional advantage of screening a collective receptor variant population is that, unlike traditional screening methods, which require that the population be segregated such that individual members can be identified, the present invention screens the receptor variant population as a non-segregated pool. The collective receptor population provides an advantage in that a collective receptor population significantly reduces the surface area or volume required to contact the collective receptor population with ligands, thereby increasing the capacity to screen many more ligands for binding interactions.

The invention provides methods for dividing the collective receptor variant population into two or more subpopulations, contacting one or more of the receptor variant subpopulations with one or more ligands and detecting one or more receptor variant subpopulations having binding activity to one or more ligands. One of the receptor variant subpopulations, all of the receptor variant subpopulations or an intermediate number of receptor variant subpopulations can be screened.

For example, a particular collective receptor population and a particular ligand or ligands can be known to give a large number of binding interactions. In this example, it is sufficient to contact a receptor variant subpopulation rather than the entire receptor variant population to identify a ligand binding to a receptor variant. One skilled in the art knows how many receptor variant subpopulations are sufficient to provide a likely probability of detecting ligand binding activity given the teachings described herein. After detecting binding of one or more ligands to a collective receptor variant population, the collective receptor variant population is divided into two or more subpopulations and contacted with the ligand or ligands. The receptor variant subpopulations can be collective when two or more receptor variants are in the subpopulation. The receptor variant subpopulations need not contain equal numbers of receptor variants. At least one of the receptor variant subpopulations will bind to the ligand or ligands, although more than one receptor variant subpopulation could be detected if more than one receptor variant binds to the ligand or ligands.

The invention also provides methods for repeating the dividing, contacting and detecting one or more times. Once binding has been detected, one or more receptor variants can be determined to have binding activity to one or more ligands. Such a determination allows identification of ligand binding activity to a receptor that can be optimal binding activity. The identification of individual receptor variants with binding to the ligand or ligands is accomplished when the receptor variant subpopulation is repeatedly divided and tested for binding activity until the receptor variant subpopulation contains only a single receptor variant that binds to one or more ligands.

Alternatively, individual receptor variants with binding to one or more ligands can be identified without dividing receptor variant subpopulations into subpopulations containing only a single receptor variant. Individual receptor variants in a collective receptor variant population can be identified using a system for tagging receptor variants. One approach is to synthesize a tag that is correlated with the generation of receptor variants. For example, a receptor variant population can be generated by mutagenizing a region of the parent receptor. While mutagenizing the receptor to generate receptor variants, a tag specific for that mutant can be generated in parallel. For example, peptides that are expressed on the surface of cells and that are recognized by specific antibodies can be used as tags to identify a co-expressed receptor variant.

Introduction of mutations that generate receptor variants can be performed, for example, using the codon-based synthesis methods described previously. Alternatively, mutations can be introduced by excising the region of the receptor cDNA to be mutagenized from a parent vector. In parallel, the region corresponding to the peptide tag can be excised as well. Mutation of a specific amino acid or amino acids in the parent receptor can be correlated with a specific mutation of one or more amino acids in the peptide to generate a unique peptide recognized by, for example, a specific antibody. The DNA fragment containing the mutated residues can be inserted into the parent vector to introduce these mutations into the receptor and the peptide tag. Appropriate restriction enzyme sites can be used to allow cloning or loxP sites can be used to allow site-specific recombination into the parent vector. Thus, a specific receptor variant is correlated with a specific peptide tag.

In the specific example of the melanophore expression system described above, a positive cell expressing a receptor variant that binds to a ligand is isolated from other cells in the population by cell sorting using dark and light properties of the melanophore cells. The isolated positive cell can then be analyzed with respect to the peptide tag expressed on its cell surface. Identification of the peptide tag allows identification of the receptor variant that binds the ligand.

A sufficiently large number of tags can be generated with a limited number of different peptides and antibodies specific for those peptides. This can be accomplished by restricting specific peptides to specific positions. For example, a combination of 32 different peptides can be used to generate 4096 (8⁴) different tags by restricting 8 specific peptides to 4 specific positions.

The tag system can be used to isolate and identify individual receptor variants in a collective receptor variant population that binds to a ligand or ligands. For example, a cell surface expressed tag consisting of peptides can be identified using antibodies specific for the peptides in fluorescence activated cell sorting (FACS) analysis. Individual receptor variants can be isolated using the unique tag associated with each receptor variant. In addition, because the tag is coordinated with a specific receptor variant, the individual receptor variant can be identified. In the case where 32 peptide and antibody combinations are used to generate 4096 different tags, exposing the cells to each of the 32 antibodies in FACS analysis allows the isolation and identification of individual receptor variants. The number of individual receptor variants that binds to the ligand or ligands can be used to identify an optimal binding ligand and can give an indication of the efficaciousness of the ligand as a lead compound for drug development.

The invention also provides a method for determining binding of a ligand to one or more receptors by contacting a collective ligand variant population with one or more receptors and detecting binding of one or more receptors to the collective ligand variant population.

The invention further provides a method for dividing the collective ligand variant population into two or more subpopulations, contacting one or more of the two or more subpopulations with one or more receptors and detecting one or more ligand variant subpopulations having binding activity to one or more receptors.

Methods and procedures described above for determining binding of a receptor to one or more ligands can similarly be applied to determine the binding of a ligand to one or more receptors. As described herein, methods are provided for repeating the dividing of ligand variant population or subpopulations, contacting with one or more receptors and detecting binding activity. Furthermore, detection of ligand binding activity allows identification of a ligand variant having binding activity to one or more receptors. Optimal binding activity can be determined relative to a predetermined standard. For example, the ligand with optimal binding can be the ligand that binds to one or more receptors at the highest affinity. Alternatively, optimal binding can be binding to the largest number of receptor variants or binding to greater than some threshold number of receptor variants.

The invention additionally provides a method for determining binding of a ligand to a receptor or variant thereof by contacting a collective ligand population with the receptor or variant thereof and detecting binding of the receptor or variant thereof to the collective ligand population.

The collective ligand population, which can be structurally related ligand variants or can be unrelated structurally, is contacted with a parent receptor or one or more receptor variants. For example, the parent receptor and receptor variants can be expressed in an appropriate cell line such as the melanophore cell line. The collective ligand population is contacted with the parent or one or more receptor variants and binding of one or more ligands in the collective ligand population is detected, for example, by detecting a change in melanophore cell color.

The invention additionally provides methods for dividing the collective ligand population into two or more subpopulations, contacting one or more of the two or more subpopulations with the receptor or variant thereof and detecting one or more ligand subpopulations with binding activity to the receptor or variant thereof. The ligand subpopulations can contain an unequal number of ligands.

The invention further provides methods for repeating the dividing, contacting and detecting one or more times. The ligand population can be divided until the subpopulation contains a single ligand. Detection of ligand binding activity allows identification of a ligand variant having binding activity to the receptor or variant thereof. An individual ligand having optimal binding activity is determined relative to a predetermined standard.

The invention also provides a method for identifying an optimal binding ligand variant for a receptor. The method consists of (a) contacting a collective receptor variant population or subpopulation thereof with a ligand population; (b) detecting binding of one or more ligands in the ligand population to the collective receptor variant population or subpopulation thereof; (c) dividing the ligand population into subpopulations; and (d) repeating optionally each of steps (a) to (c), wherein the ligand subpopulation in step (c) comprises two or more ligands and is used as the ligand population in step (a) and wherein the detecting in step (b) identifies one or more ligands having binding activity to the collective receptor variant population.

The method for identifying an optimal binding ligand variant can include the additional steps of (e) generating a library of variants of the ligand identified in step (d); (f) contacting a parent receptor with each of the ligand variants; and (g) detecting the binding of one or more ligand variants to the parent receptor.

Following identification of one or more ligands having binding activity to the collective receptor variant population, the identified ligand can be used as a parent ligand to generate a library of ligand variants with structural similarities to the parent ligand. The library of ligand variants can be, for example, a population of ligand variants that are screened for binding activity to the parent receptor. Once ligand variants having binding activity have been identified, the binding activity of the ligand variants can be further compared to each other or to a predetermined standard. Such a comparison allows identification of a ligand variant having optimal binding activity to a parent receptor.

As described previously in regard to the multiple binding points of reference for ligand-receptor interactions, particular chemical functional groups can be fixed so that they are identical to the parent ligand. Ligand variants with one chemical group fixed differ from the parent ligand at other chemical groups. Following identification of a ligand with optimal binding, a library of ligand variants can be generated and a ligand variant having optimal binding to the parent receptor is determined. The ligand variant with optimal binding to the parent ligand can be used as a second parent ligand to generate a second library of ligand variants. Such ligand variants can have two chemical groups fixed to be identical to the second parent ligand. An iterative process of identifying individual ligands or ligand variants with optimal binding to the parent receptor and generating a new library based on that identified ligand variant can be repeated to determine a ligand variant with optimal binding to the parent receptor. The ligand variants can be identified based on structural or functional criteria or synthesized by various means known to those skilled in the art. Where the ligand is a polypeptide, for example, variants can be made and screened using surface display methods known to those skilled in the art and using, for example, the codon-based synthesis procedures described previously.

The invention also provides a method for identifying an optimal binding ligand variant to a receptor. The method consists of (a) contacting two or more subpopulations of a collective receptor variant population with individual ligands from a ligand population; (b) detecting binding of one or more individual ligands to one or more of the subpopulations of the collective receptor variant population; (c) dividing at least one of the subpopulations of the collective receptor population which exhibits binding activity to the individual ligands into two or more new subpopulations; and (d) repeating optionally each of steps (a) to (c), the two or more new subpopulations in step (c) comprising two or more receptor variants and the new subpopulations used as the two or more subpopulations of a collective receptor variant population in step (a), wherein the detecting in step (b) identifies one or more individual ligands having binding activity to one or more new subpopulations of subpopulations of the collective receptor variant population.

The method for identifying an optimal binding ligand variant can include the additional steps of (e) contacting a closely related receptor variant subpopulation comprising a parent receptor or a closely related variant thereof with one or more individual ligands identified in step (d); (f) detecting binding of one or more individual ligands to the closely related receptor variant subpopulation; and (g) comparing the binding activity of one or more ligands having binding activity to the closely related receptor variant subpopulation, wherein said comparing identifies a ligand having optimal binding activity to the closely related receptor variant subpopulation.

The method for identifying an optimal binding ligand variant to a receptor can also include the additional steps of (h) generating a library of variants of said ligand identified in step (g); (i) contacting said parent receptor with each of said ligand variants; and (j) detecting binding of one or more ligand variants to said parent receptor.

After identifying one or more ligands having binding activity to the collective receptor variant population, the identified one or more ligands can be further used to screen a closely related receptor variant subpopulation containing at least a parent receptor or a closely related variant thereof. The subpopulation can contain any number of receptor variants so long as they are closely related to the parent receptor. One skilled in the art knows the closeness of the relationship of the receptor variants to the parent receptor sufficient to determine an optimal binding ligand. A ligand that binds to the most number of receptor variants in a closely related receptor variant subpopulation will have the greatest probability of binding to the parent receptor and has the greatest likelihood of being an optimal binding ligand. Such an optimal binding ligand can be used as a lead compound for drug development. In contrast, a receptor variant subpopulation containing less closely related receptor variants provides a decreased probability that a ligand that binds to the most number of receptor variants will also bind to the parent receptor.

A ligand having optimal binding activity to the closely related receptor variant subpopulation can be further used as a parent ligand to generate a library of ligand variants with structural similarities to the parent ligand. One skilled in the art knows what optimal binding activity is desired. For example, a ligand having optimal binding activity can be one that binds to the most number of receptor variants in the closely related receptor variant subpopulation. Optimal binding activity also can be defined as ligands that bind to a minimum threshold of numbers of receptor variants. The library of ligand variants can be, for example, a population of ligand variants that are screened for binding activity to the parent receptor. Once ligand variants having binding activity have been identified, the binding activity of the ligand variants can be compared to each other or to a predetermined standard. Such a comparison allows identification of a ligand variant having optimal binding activity to a parent receptor.

It is understood that modifications which do not substantially affect the activity of the various embodiments of this invention are also provided within the definition of the invention provided herein. Accordingly, the following examples are intended to illustrate but not limit the present invention.

### EXAMPLE I

### Preparation of Melanophore Cells Expressing a Receptor Variant Population

This example demonstrates expression of a polypeptide receptor variant population in melanophore cells and screening ligands for binding activity.

Frog melanophore cells derived from Xenopus laevis were grown in conditioned frog media at 27° C. Conditioned frog media was made by growing frog fibroblasts in Leibovitz L-15 media (0.5× concentration) containing 20% heat inactivated fetal calf serum for 4 days, collecting the media supernatant from the fibroblasts and filtering the supernatant through a 0.2 μ m filter. Frog melanophore cell cultures were periodically centrifuged through PERCOLL density gradients to enrich for more highly pigmented cells. Briefly, cells were trypsinized, suspended in quench frog media containing Leibovitz L-15 media (0.5× concentration) with 20% calf serum and centrifuged at 1500 rpm for 5 min. Cells were resuspended in 20% PERCOLL, 80% quench frog media. Cells were layered onto 2 volumes of 50% PERCOLL, 50% quench frog media and centrifuged at 600-800 rpm for 10 min. The supernatant was aspirated and cells were resuspended in quench frog media and the cells were transferred to a new tube and centrifuged at 1500 rpm for 5 min. The pellets contained melanophore cells enriched for more highly pigmented cells.

A receptor variant population is generated by identifying a region of a receptor cDNA that encodes a ligand binding site of interest. The ligand binding site of interest is excised from a parental vector using methods well known to those skilled in the art (Sambrook et al, 1989, supra). The excised fragment is used to introduce mutations in the ligand binding domain of the receptor. Mutant oligonucleotides are generated to introduce specific mutations into the ligand binding domain. Following mutagenesis, DNA corresponding to mutant ligand binding domains are introduced back into the parental vector to generate receptor variants.

Tags specific for each receptor variant also are generated. For coexpression of a receptor variant and a peptide tag, both the receptor and peptide tag are present on the parental expression vector. In parallel to excision of the ligand binding domain for mutagenesis, the DNA encoding the peptide tag is excised as well. Mutant oligonucleotides are synthesized to introduce a mutation or mutations into the receptor and simultaneously introduce a mutation or mutations into the tag. Upon introducing the mutated DNA back into the parental vector, a receptor variant is generated with a correlated tag expressed on the cell surface. Each tag is composed of specific combinations of peptides that are recognized by distinct antibodies. The antibodies are used to identify the receptor variant correlated with that tag.

Melanophore cells are transfected using electroporation (Potenza et al., Anal. Biochem. 206:315-322 (1992)). In addition, other methods well known to those skilled in the art can be used to transfect melanophores (Sambrook et al., 1989, supra). Expression of transfected proteins are assessed 2 to 3 days following transfection. Stable cell lines expressing transfected proteins can be obtained by treating cells under the appropriate selection conditions or with the appropriate drug. To minimize clonal variation, a melanophore cell line is generated that contains a chromosomally integrated neo gene for selection of neomycin resistance using G418. A loxP site is located at the 5′ end of the neo gene, but the gene has no promoter. The parental expression vector contains receptor or receptor variant DNA with its own promoter as well as a downstream promoter 3′ of the receptor DNA. LoxP sites are located at the 5′ end of the receptor DNA and at the 3′ end of the downstream promoter. The receptor or receptor variant DNA is transfected into cells and site-specific recombination occurs at the loxP sites. When site specific recombination at the loxP sites occurs, the downstream promoter is placed at the 5′ end of the neo gene, thus providing a selectable marker and an indication that site-specific recombination and introduction of the receptor or receptor variant DNA into the cells has occurred. An advantage of this loxP system is that the receptor or receptor variant is introduced into the same location in the melanophore cell genome, thus minimizing clonal variation due to different sites of integration in the genome.

Melanophore cells expressing a collective receptor variant population are plated into one or more microtiter wells. Cells are treated with one or more ligands either as individual ligands are as pools of ligand subpopulations. Ligand binding is determined by testing the effect of ligands on signaling by the receptor variants. Phototransmission at 620 nm is measured to determine those wells which are positive for ligand binding to the collective receptor population.

Following the determination of positive ligand binding, the receptor variant population can be divided into subpopulations. The subpopulations are tested for positive ligand binding. In addition, individual receptor variants can be identified using its unique coexpressed tag. Cells positive for ligand binding are segregated from non-binding receptor variants by cell sorting using the light and dark properties of the melanophores. The segregated positive cells are sequentially exposed to each antibody used to identify the peptides in each receptor variant tag for sorting cells by fluorescence activated cell sorting using a Becton Dickinson FACSort system. Cells are initially subdivided into cells that react with one or more specific antibodies before determining the unique antibody combination that identifies each individual receptor variant. The number of individual receptor variants that bind to a given ligand are determined. The specific mutations associated with the ligand binding receptor variants also are determined by correlating the unique tag with the mutation of specific residues in the parent receptor.

These results demonstrate the generation of a receptor variant population correlated with identifiable tags and the identification of a ligand with optimal binding activity.

### EXAMPLE II

### The Probability of Binding a Focused Library and a Diverse Library of Ligands to a Receptor

This example demonstrates the probability of binding a focused library and a diverse library of ligands to a receptor.

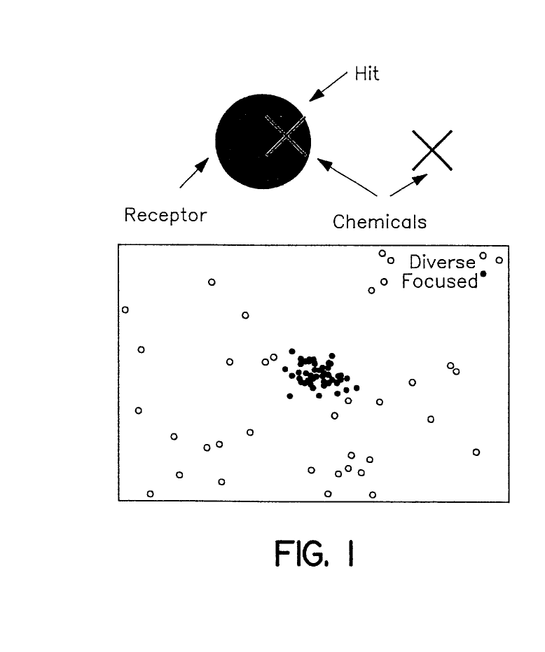

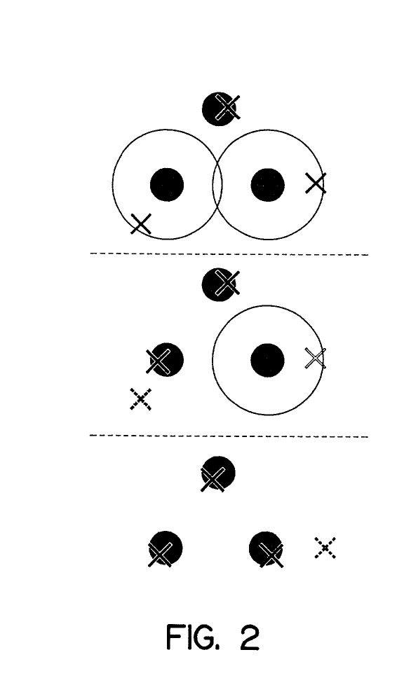

A ligand is represented as a point in space and a receptor is represented as a disc in space. A ligand binds to a receptor when the ligand lies inside the disc corresponding to the receptor (corresponding to “hit” in FIG. 1).

A ligand variant population, represented as points in space, is generated by selecting ligand variants uniformly and randomly such that the ligand variants form a distribution such as a Gaussian distribution around the parent ligand, represented as a point in space. This is accomplished by varying the chemical functional groups on the parent ligand. The closer the ligand variants fall relative to the parent ligand, the more similar the variants are chemically to the parent ligand. This is represented as the relative closeness of the points representing the ligand variants to the center of a Gaussian distribution around the point representing the parent ligand. The parameter selected to determine the Gaussian distribution of the ligand variants around the parent ligand provides a given probability of a ligand variant binding to a receptor.

Similarly, a receptor variant population, represented as discs in space, is generated by selecting receptor variants uniformly and randomly around the center of the disc in space representing the parent receptor such that the receptor variants form a distribution such as a Gaussian distribution around the parent receptor. This is accomplished by varying the chemical functional groups on the parent receptor. The closer the receptor variants fall relative to the parent receptor, the more similar the variants are chemically to the parent receptor. This is represented as the relative closeness of the points representing the receptor variants to the center of a Gaussian distribution around the center of the disc representing the parent receptor. The parameter selected to determine the Gaussian distribution of the receptor variants around the parent receptor provides a given probability that a ligand that binds to a receptor variant will also bind to the parent receptor.

The distribution of ligands and receptors is generally chosen so that the distribution of receptors is smaller than the distribution of ligands. In this case, the variance around the receptor is relatively small, reflecting receptor variants closely related to the parent receptor. Choosing the distribution of receptors to be smaller than the distribution of ligands increases the probability that a ligand that binds to the receptor variants will also bind to the parent ligand.

In a diverse library of ligands, the ligands are distributed over a large area (see FIG. 1, bottom panel). The probability of a given ligand binding to a receptor represented as a disc in that area is decreased because there are larger gaps between the ligands. The larger gaps between ligands represent diversity of chemical functional groups of the ligands. However, there is a greater probability of binding to a larger number of receptors since the ligands are dispersed over a larger area.

In contrast to a diverse library, a focused library of ligands has ligands distributed in a smaller area due to the fact that the ligands are more closely related (see FIG. 1, bottom panel). While the probability of focused ligands binding to a variety of receptors is low due to the ligands being in a smaller area, the probability that more of the focused ligands will bind to a given receptor is high when that receptor coincides with the focused ligands. For example, if a disc representing a receptor was centered over the area covered by the focused ligands shown in FIG. 1, a number of ligands would bind to the receptor. However, the same receptor centered over the focused ligands would bind very few, if any, of the diverse ligands. Therefore, the type of ligand library is determined by the particular goals of the screen.

These results demonstrate that using a diverse library of ligands increases the probability of finding a ligand that binds to any receptor. In contrast, using a focused library of ligands increases the probability of finding a ligand that binds to a given receptor. Thus, predictions can be made as to the likelihood of identifying a ligand variant that binds to a receptor.

### EXAMPLE III

### The Probability of Identifying a Ligand that Binds a Receptor Depends on Molecular Interactions

This example demonstrates that the probability of identifying a ligand that binds a receptor depends on molecular interactions.

Binding of a ligand to a receptor generally occurs through a series of smaller interactions resulting from multiple contact points or through multiple interactions of a chemical functional group. To describe molecular interactions in a ligand-receptor binding interaction, a ligand is represented as three points in space and a receptor is represented as three discs in space. The three points representing the ligand correspond to three molecular interactions occurring through chemical groups on the ligand that serve as contact points for receptor binding. Similarly, the three discs representing the receptor correspond to three molecular interactions occurring through chemical groups on the receptor that serve as contact points for ligand binding. A ligand binds to a receptor when three points of the ligand lie inside the three discs corresponding to the receptor.

As described in Example II, parameters are selected to determine the Gaussian distribution of ligand variants around the three points representing the parent ligand. Similarly, parameters are selected to determine the Gaussian distribution of receptor variants around the three discs representing the parent receptor. In this case, the distribution around each point of the parent ligand or each disc of the parent receptor can be varied independently. For example, one point can be held to be identical to the parent molecule while the other two points are varied. Also, the distribution around the points being varied can differ from each other.

By describing a ligand-receptor binding interaction as multiple molecular interactions, an optimal binding ligand can be identified more rapidly. For example, if one of the discs representing the parent receptor is fixed to be identical to the parent receptor while the other two disc are varied to represent receptor variants, then any ligand that binds this receptor variant has an increased likelihood of binding to the parent receptor (see FIG. 2, upper panel). The increased probability of binding to the parent receptor is determined by the fact that one of the molecular interaction sites is identical to the parent. If all three discs of the receptor parent were varied, the receptor variant would be less closely related to the parent and ligands which bind to that variant have a decreased probability of binding to the parent. Fixing one molecular interaction site to be identical to the parent generates receptor variants that are more closely related to the parent. Similarly, fixing two molecular interaction sites generates receptor variants that are even more closely related to the parent receptor (see FIG. 2, middle panel).

Using a multi-point molecular interactions representation of ligand-receptor interactions provides increased probability of identifying an optimal binding ligand. For example, focused ligands can be determined in an iterative process. In a first round of screening, a receptor variant population is generated by fixing one of the three discs representing the receptor. An optimal binding ligand identified by such a screen can be used to generate a focused library of ligands. A new receptor variant population is generated by fixing two of the discs representing the receptor. This new receptor variant population is more closely related to the parent receptor. Screening the new receptor variant population with the focused library of ligands will have greatly increased probability of identifying a ligand variant with optimal binding to the parent receptor (see FIG. 2, lower panel).

These results demonstrate that considering multi-point molecular interactions in ligand-receptor binding interactions provides rapid determination of an optimal binding ligand.

### EXAMPLE IV

### The Probability of Identifying a Binding Ligand Using a Vector Representation of Ligand-Receptor Binding Interactions

This example demonstrates that a ligand and receptor binding interaction can be described as a multi-point, spatially related interaction represented as vectors.

The chemical functional groups of the ligand and the receptor are represented as vectors rather than as points and discs in space. The length of the vectors are shorter when the molecule is smaller. Therefore, smaller molecules such as organic chemicals have shorter vectors than larger molecules such as polypeptides. Each different chemical group of the ligand and receptor is represented by distinct vectors. Therefore, each ligand or ligand variant is represented by a unique string of vectors and each receptor or receptor variant is represented by a unique string of vectors.

The binding sites of a given receptor variant or ligand variant are represented by three points. The first point is the origin of the vector string. The second point is determined by starting at the origin and summing the vectors corresponding to the positions in the first half of the string. The third point is determined by starting at the second point and summing up the vectors corresponding to positions in the second half of the string. These three points define a triangle that represents each ligand or ligand variant and receptor or receptor variant. Variant molecules with similar vector strings are more closely related since they are the sum of many of the same vectors.

Binding of a ligand to a receptor is determined if the triangle representing the ligand and the triangle representing the vector can be arranged so that the points of the two triangles are close. The closeness of the triangles is measured by determining whether the lengths of the sides of the triangles representing the ligand and receptor differ by at most some threshold value. Thus, the ability of chemical groups of a ligand to bind to chemical groups of a receptor is accounted for in the vector representation as well as the spatial relationship between chemical groups of the ligand and the chemical groups of the receptor that represent binding sites.

Random noise can be introduced to represent movements of functional groups such as small changes in the relative positions of chemical groups in the molecules. In addition, random noise can be introduced to represent unknown parameters that affect ligand-receptor interactions.

To represent ligands and receptors, parameters are determined for the length of vector strings, the size of the vectors, the number of different chemical groups accounted for, the probability of a large change, the size of the random noise and the threshold for closeness of lengths of triangle sides.

The probability of finding a binding partner is determined by the variance chosen for the vectors. A high probability of finding a binding partner is provided when the vector is chosen to have small variance, which represents variants that are closely related to a parent molecule. A smaller probability of finding a binding partner is provided when the vector is chosen to have large variance, which represents variants that are more distantly related to a parent molecule. For example, when one of the binding molecules is a small molecule, the lengths of the vectors are small. If the binding partners are large molecules, the lengths of the vectors are large. Therefore, to generate a triangle with sidelengths of a similar size between large and small binding partners, a larger variance is introduced into the small molecule to increase the probability of its binding to the large molecule. In an example where a ligand is a small molecule and a receptor is a large molecule, the greatest probability of finding a binding ligand occurs when the receptor variants are closely related, represented by vectors with small variance, and the ligands are less closely related, represented by vectors with large variance. This occurs because small molecules are represented by a small number of small vectors. In order to sum this smaller number of small vectors to obtain triangle sidelengths of similar size to a large molecule, a large variance in the vectors representing the small molecule is introduced.

These results show that ligands and receptors can be represented as vectors to determine the probability of identifying a ligand that binds to a receptor.

### EXAMPLE V

### Optimization of Anti-Idiotypic Antibody Ligands

This example shows that screening ligands with receptor variants increases the probability of identifying an optimal binding ligand.

The parent receptor was antibody BR96, a mouse monoclonal antibody to Le^{Y}-related cell surface antigens. Six receptor variants were generated using random codon synthesis as described in U.S. Pat. No. 5,264,563 and in Glaser et al. supra. Briefly, synthesis was performed using two DNA synthesizer columns. For simplicity, the DNA sequences are referred to as the coding strand although, in practice, all oligonucleotides were synthesized as the complementary sequence. On column 1 a trinucleotide coding for the predetermined parental codon found at the CDR positions specified below was synthesized. On column 2 a random codon encoding all 20 amino acids was synthesized using the nucleotides XXG/T where X represents a mixture of dA, dG, dC and T cyanoethyl phosphoramidites. The use of the XXG/T codon reduces the number of stop codons to include only UAG, which can be suppressed in supE E. coli bacterial strains. After synthesis of each codon, the beads from the two columns were mixed together, divided in half, and then repacked into two new columns. The columns were then returned to the DNA synthesizer and the process was repeated for the subsequent CDR positions. After the final synthesis step the contents of the two columns were pooled and the resulting oligonucleotides purified. This particular application of codon-based synthesis results in a mixture of oligonucleotides coding for randomized amino acids within a predefined region while maintaining a 50% bias toward the parental sequence at any position. By altering the proportion of the beads in the two columns, the level of substitution with respect to parental sequence can be further controlled. Furthermore, any given position can retain a specified codon and mixtures of codons other than XXG/T can be used to insert only some subset of amino acid residues if desired.

Oligonucleotides containing randomized codons were used to generate receptor variants by mutagenesis (Kunkel, Proc. Natl. Acad. Sci. USA 82:488-492 (1985) and Kunkel et al., Methods Enzymol. 154:367-382 (1987)). Briefly, M13IXL604 or M13IXL605 phage were grown in the dut⁻ ung⁻ Escherichia coli strain CJ236 (BioRad, Richmond, Calif.) and phage were precipitated by adding 0.25 volumes of 3.5 M ammonium acetate, 20% polyethylene glycol/ml of cleared culture supernatant. Uracil-substituted single stranded DNA was isolated by phenol extraction followed by ethanol precipitation. From 6 to 8 pmol of phosphorylated oligonucleotide were used to mutagenize 250 ng of the chimeric L6 template in a 13 μl reaction volume (Huse et al., J. Immunol. 149:3914-3920 (1992). The reaction products were diluted twofold with water and 1 μl was electroporated into E. coli strain XL-1 (Stratagene, San Diego, Calif.) and titered onto a lawn of XL-1.

Three anti-idiotypic antibody ligands were generated by immunizing 6 or 7-week-old BALB/c mice intraperitoneal (four times, once every 20 days) with 50 μg of purified antibody BR96 using aluminum hydroxide as adjuvant. The reactivity of the mice sera was tested by ELISA (Fields et al., Nature 374:739-742 (1995)). After a final boost with soluble polyclonal rabbit IgG, mice with the strongest response were killed and the spleens were used to obtain hybridomas as described (Galfre and Milstein, Methods Enzymol. 73:3-46 (1981)).

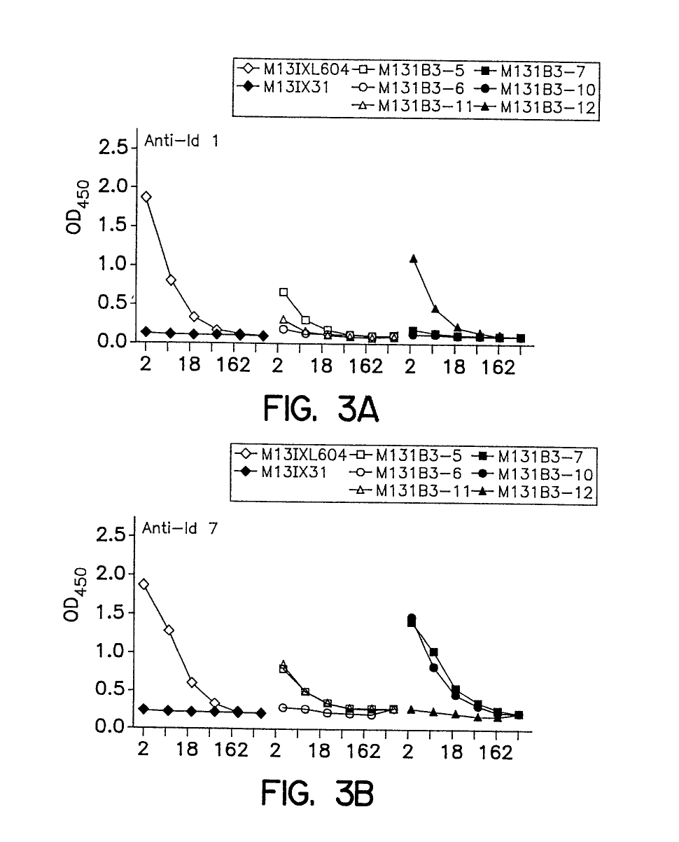

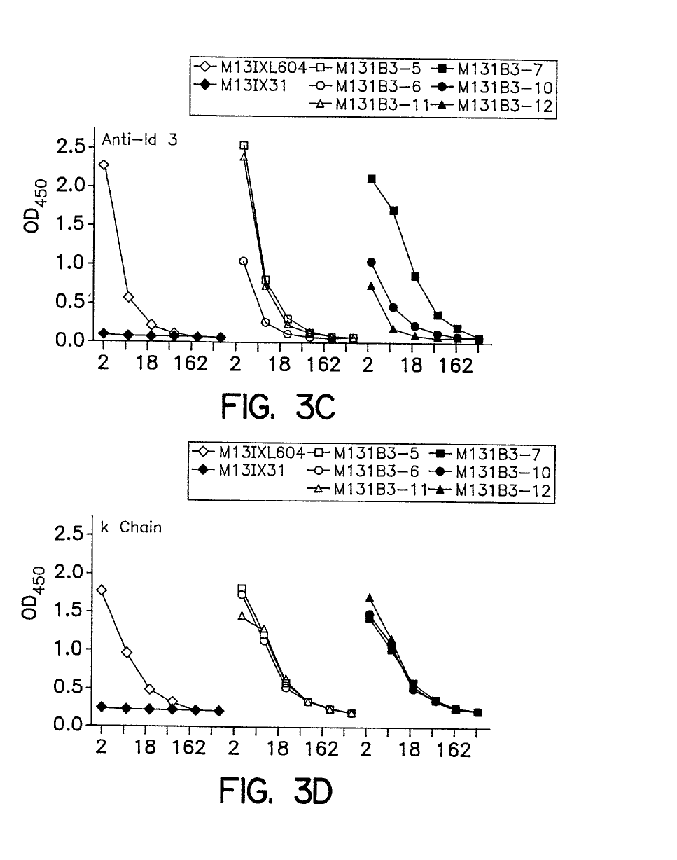

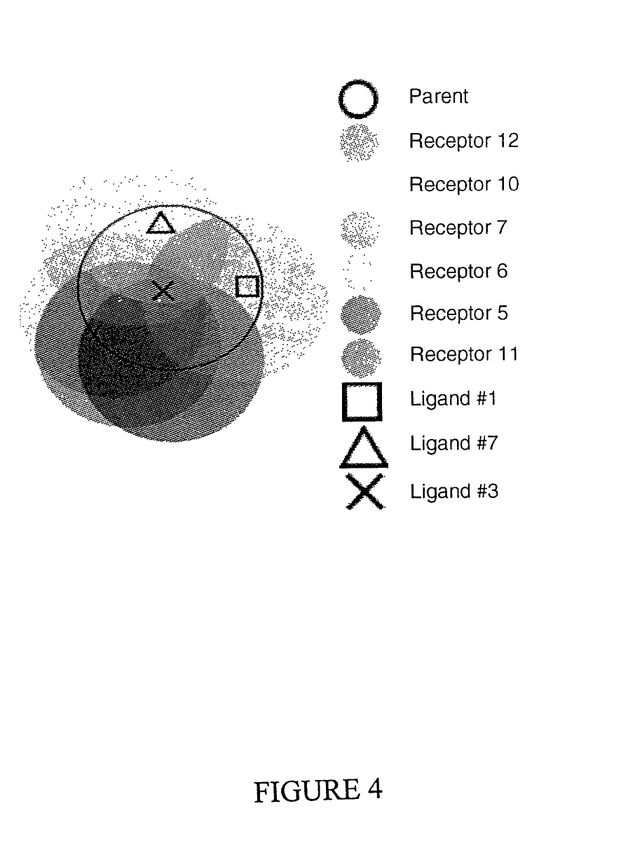

Receptor variants were screened for binding to anti-idiotypic antibody ligands. The anti-idiotypic antibody ligands were screened against the parent receptor and six receptor variants to determine binding activity using an ELISA assay (see FIG. 3). Anti-idiotypic antibody No. 1 was classified as binding to receptor 12 and the parent receptor. Anti-idiotypic antibody No. 7 was classified as binding to receptor 7, receptor 10 and the parent receptor. Anti-idiotypic antibody No. 3 was classified as binding to all of the receptors, including the parent receptor.

The nucleotide and amino acid sequences of the light chain CDR regions 1 and 2 of the parent receptor (designated wild type) and the six receptor variants (designated M131B3-5 through M131B3-12) are shown in Table I. The nucleotide and amino acid sequences (SEQ ID NOS: 1, 3, 5, 7, 9, 11, 13, and 2, 4, 6, 8, 10, 12, 14, respectively) for the CDR L1 region of the parent and six receptor variants are shown in the top half of Table I. The nucleotide and amino acid sequence (SEQ ID NOS: 15, 17, 19, 21, 23, 25, 27 and 16, 18, 20, 22, 24, 26, 28, respectively) for the CDR L2 region of the parent and six receptor variants are shown in the bottom half of Table I. In Table I, L1 and L2 CDR mutations in M13IXL604 clones were selected on the basis of binding to anti-idiotypic antibody No. 3 similar to that of wild type and negligible binding to anti-idiotypic antibody No. 1. Changes resulting from the mutagenesis procedure are indicated by boldface type.

Several positions in the receptor sequence were found to be conserved while other positions were found to differ from the parent receptor in both CDR regions 1 and 2. Substitutions occurred at all five target loci in CDR L1 and at three loci in CDR L2. The total number of substitutions in CDR L1 and CDR L2 ranged from two to four in each mutant.

TABLE I
Nucleotide and Amino Acid Sequences of Receptor
Variants of BR96 Antibody

| | | | | | | | | |
|---|---|---|---|---|---|---|---|---|
| Amino Acid | 26 | 27 | 28 | 29 | 30 | 31 | 32 | 33 |
| CDR L1 | | | | | | | | |
| Wild type | AGC | TCA | AGT | GTA | AGT | TTC | ATG | AAC |
| | Ser | Ser | Ser | Val | Ser | Phe | Met | Asn |
| M131B3-5 | AGC | TCA AGT | GTA | AGG | TTC | ATG | AAC | |
| | Ser | Ser | Ser | Val | Arg | Phe | Met | Asn |
| M131B3-G | AGC | GAG | AGT | GTA | AAT | CTT | ATG | AAC |
| | Ser | Glu | Ser | Val | Asn | Leu | Met | Asn |
| M131B3-7 | AGC | TCA | AGT | GTT | AAT | TTC | ATG | AAC |
| | Ser | Ser | Ser | Val | Asn | Phe | Met | Asn |
| M131B3-10 | AGC | TCA | ACG | GTA | AGT | TTC | ATG | AAC |
| | Ser | Ser | Thr | Val | Ser | Phe | Met | Asn |
| M131B3-11 | AGC | TCA | AGT | GTA | GCG | TAT | ATG | AAC |
| | Ser | Ser | Ser | Val | Ala | Tyr | Met | Asn |
| M131B3-12 | AGC | CAG | AGT | GCT | AAG | CAT | ATG | AAC |
| | Ser | Gln | Ser | Ala | Lys | His | Met | Asn |
| Amino Acid | 49 | 50 | 51 | 52 | 53 | 54 | 55 | 56 |
| CDR L2 | | | | | | | | |
| Wild type | GCC | ACA | TCC | AAT | TTG | GCT | TCT | GGA |
| | Ala | Thr | Ser | Asn | Leu | Ala | Ser | Gly |
| M131B3-5 | GCC | ACA | GAG | AAG | TTG | GCT | TCT | GGA |
| | Ala | Thr | Glu | Lys | Leu | Ala | Ser | Gly |
| M131B3-6 | GCC | ACA | GTT | AAT | TTG | GCT | TCT | GGA |
| | Ala | Thr | Val | Asn | Leu | Ala | Ser | Gly |
| M131B3-7 | GCC | ACA | GTG | AAT | TTG | GCT | TCT | GGA |
| | Ala | Thr | Val | Asn | Leu | Ala | Ser | Gly |
| M131B3-10 | GCC | ACA | TCC | AGG | GCG | GCT | TCT | GGA |
| | Ala | Thr | Ser | Arg | Ala | Ala | Ser | Gly |
| M131B3-11 | GCC | ACA | CAG | AAT | TTG | GCT | TCT | GGA |
| | Ala | Thr | Gln | Asn | Leu | Ala | Ser | Gly |
| M131B3-12 | GCC | ACA | TCC | AAT | TTG | GCT | TCT | GGA |
| | Ala Thr | Ser | Asn | Leu | Ala | Ser | Gly | |

The results of the screen are summarized in FIG. 6, where receptors are represented as discs and ligands are represented as symbols. These results demonstrate that screening ligands against a population of receptor variants will rapidly identify ligands having optimal binding activity. For example, if the collective receptor variant population of this example were screened in the melanophore system, ligand No. 3 would have generated the highest signal since it binds to all seven receptors in the receptor variant population. Ligand No. 7 would give a weaker signal since this ligand binds to three receptors in the receptor variant population. Ligand No. 1 would give a still weaker signal since this ligand binds to two receptors in the receptor variant population. Thus, screening with a collective receptor variant population provides more information about the binding characteristics of the ligand than screening with the parent receptor alone. In addition, ligands that bind weakly to the parent receptor may not have been detectable above background when screened against the parent alone but are detectable when more than one receptor in the receptor variant population binds to the ligand.

These results demonstrate that screening a receptor variant population rapidly identifies optimal binding ligands to a receptor.

Throughout this application various publications have been referenced within parentheses. The disclosures of these publications in their entireties are hereby incorporated by reference in this application in order to more fully describe the state of the art to which this invention pertains.

Although the invention has been described with reference to the examples provided above, it should be understood that various modifications can be made without departing from the spirit of the invention. Accordingly, the invention is limited only by the claims.

I claim:

1. A method for determining binding of a receptor to one or more ligands, comprising contacting a collective receptor variant population with said one or more ligands and detecting binding of said one or more ligands to said collective receptor variant population.

2. The method of claim 1, further comprising dividing said collective receptor variant population into two or more subpopulations, contacting one or more of said two or more subpopulations with said one or more ligands and detecting one or more receptor variant subpopulations having binding activity to said one or more ligands.

3. The method of claim 2, wherein said dividing, contacting and detecting are repeated one or more times.

4. The method of claim 3, wherein said detecting identifies a receptor variant having binding activity to said one or more ligands.

5. The method of claim 4, wherein said detecting identifies a receptor variant having optimal binding activity to said one or more ligands.

6. The method of claim 1, wherein said receptor variant population is recombinantly expressed in cells.

7. The method of claim 6, wherein said cells are melanophores.

8. The method of claim 1, further comprising dividing said collective receptor variant population into two or more subpopulations, contacting said two or more subpopulations with said one or more ligands and detecting one or more receptor variant subpopulations having binding activity to said one or more ligands.

9. The method of claim 1, further comprising isolating an individual receptor variant having binding activity to said one or more ligands, wherein said receptor variant is linked to an identifiable tag.

10. A method for determining binding of a ligand to one or more receptors, comprising contacting a collective ligand variant population with said one or more receptors and detecting binding of said one or more receptors to said collective ligand variant population.

11. The method of claim 10, further comprising dividing said collective ligand variant population into two or more subpopulations, contacting one or more of said two or more subpopulations with said one or more receptors and detecting one or more ligand variant subpopulations having binding activity to said one or more receptors.

12. The method of claim 11, wherein said dividing, contacting and detecting are repeated one or more times.

13. The method of claim 12, wherein said detecting identifies a ligand variant having binding activity to said one or more receptors.

14. The method of claim 13, wherein said detecting identifies a ligand variant having optimal binding activity to said one or more receptors.

15. The method of claim 10, wherein said ligand variant population is recombinantly expressed in cells.

16. The method of claim 15, wherein said cells are melanophores.

17. The method of claim 10, further comprising isolating an individual ligand variant having binding activity to said one or more ligands, wherein said ligand variant is linked to an identifiable tag.

18. The method of claim 10, further comprising dividing said collective ligand variant population into two or more subpopulations, contacting said two or more subpopulations with said one or more receptors and detecting one or more ligand variant subpopulations having binding activity to said one or more receptors.

19. A method for determining binding of a ligand to a receptor or a variant thereof, comprising contacting a collective ligand population with said receptor or variant thereof and detecting binding of said receptor or variant thereof to said collective ligand population.

20. The method of claim 19, further comprising dividing said collective ligand population into two or more subpopulations, contacting one or more of said two or more subpopulations with said receptor or variant thereof and detecting one or more ligand subpopulations having binding activity to said receptor or variant thereof.

21. The method of claim 20, wherein said dividing, contacting and detecting are repeated one or more times.

22. The method of claim 21, wherein said detecting identifies a ligand variant having binding activity to said receptor or variant thereof.

23. The method of claim 22, wherein said detecting identifies a ligand variant having optimal binding activity to said receptor or variant thereof.

24. The method of claim 19, wherein said collective ligand population contains ligand variants.

25. The method of claim 19, further comprising dividing said collective ligand population into two or more subpopulations, contacting said two or more subpopulations with said receptor or variant thereof and detecting one or more ligand subpopulations having binding activity to said receptor or variant thereof.

26. A method for identifying an optimal binding ligand variant for a receptor, comprising:
(a) contacting a collective receptor variant population or subpopulation thereof with a ligand population;
(b) detecting binding of one or more ligands in said ligand population to said collective receptor variant population or subpopulation thereof;
(c) dividing said ligand population into subpopulations; and
(d) repeating optionally each of steps (a) to (c), wherein said ligand subpopulation in step (c) comprises two or more ligands and is used as said ligand population in step (a) and wherein said detecting in step (b) identifies one or more ligands having binding activity to said collective receptor variant population.

27. The method of claim 26, further comprising the steps:
(e) generating a library of variants of said ligand identified in step (d);
(f) contacting a parent receptor with each of said ligand variants; and
(g) detecting the binding of one or more ligand variants to said parent receptor.

28. The method of claim 26, wherein step (d) further comprises comparing the binding activity of said one or more ligands having binding activity to said receptor variant population.

29. The method of claim 28, wherein said comparing identifies a ligand having optimal binding activity to said collective receptor variant population.

30. The method of claim 27, wherein said step (g) further comprises comparing the binding activity of said one or more ligand variants having binding activity to said parent receptor.

31. The method of claim 30, wherein said comparing identifies a ligand having optimal binding activity to said parent receptor.

32. A method for identifying an optimal binding ligand variant to a receptor, comprising:
(a) contacting two or more subpopulations of a collective receptor variant population with individual ligands from a ligand population;
(b) detecting binding of one or more individual ligands to one or more of said subpopulations of said collective receptor variant population;
(c) dividing at least one of said subpopulations of said collective receptor population which exhibits binding activity to said individual ligands into two or more new subpopulations; and
(d) repeating optionally each of steps (a) to (c), said two or more new subpopulations in step (c) comprising two or more receptor variants and said new subpopulations used as said two or more subpopulations of a collective receptor variant population in step (a), wherein said detecting in step (b) identifies one or more individual ligands having binding activity to one or more new subpopulations of subpopulations of said collective receptor variant population.

33. The method of claim 32, further comprising the steps:
(e) contacting a closely related receptor variant subpopulation comprising a parent receptor or a closely related variant thereof with one or more individual ligands identified in step (d);
(f) detecting binding of said one or more individual ligands to said closely related receptor variant subpopulation; and
(g) comparing the binding activity of said one or more ligands having binding activity to said closely related receptor variant subpopulation, wherein said comparing identifies a ligand having optimal binding activity to said closely related receptor variant subpopulation.

34. The method of claim 33, further comprising the steps:
(h) generating a library of variants of said ligand identified in step (g);
(I) contacting said parent receptor with each of said ligand variants; and
(j) detecting binding of one or more ligand variants to said parent receptor.

35. The method of claim 32, wherein step (d) further comprises comparing the binding activity of said one or more ligands having binding activity to said closely related receptor variant population.

36. The method of claim 35, wherein said comparing identifies a ligand having optimal binding activity to said collective receptor variant population.

37. The method of claim 34, wherein said step (j) further comprises comparing the binding activity of said one or more ligand variants having binding activity to said parent receptor.

38. The method of claim 37, wherein said comparing identifies a ligand having optimal binding activity to said parent receptor.


SEQUENCE LISTING (28 sequences)

SEQ ID NO: 1 - DNA, 24 bp, Mus musculus
  CDS (1)..(24)
agctcaagtgtaagtttcatgaac

SEQ ID NO: 2 - protein, 8 aa, Mus musculus
SSSVSFMN

SEQ ID NO: 3 - DNA, 24 bp, Artificial Sequence
  CDS (1)..(24): Description of Artificial Sequence synthetic construct
agctcaagtgtaaggttcatgaac

SEQ ID NO: 4 - protein, 8 aa, Artificial Sequence
  Description of Artificial Sequence synthetic
SSSVRFMN

SEQ ID NO: 5 - DNA, 24 bp, Artificial Sequence
  CDS (1)..(24): Description of Artificial Sequence synthetic construct
agcgagagtgtaaatcttatgaac

SEQ ID NO: 6 - protein, 8 aa, Artificial Sequence
  Description of Artificial Sequence synthetic
SESVNLMN

SEQ ID NO: 7 - DNA, 24 bp, Artificial Sequence
  CDS (1)..(24): Description of Artificial Sequence synthetic construct
agctcaagtgttaatttcatgaac

SEQ ID NO: 8 - protein, 8 aa, Artificial Sequence
  Description of Artificial Sequence synthetic
SSSVNFMN

SEQ ID NO: 9 - DNA, 24 bp, Artificial Sequence
  CDS (1)..(24): Description of Artificial Sequence synthetic construct
agctcaacggtaagtttcatgaac

SEQ ID NO: 10 - protein, 8 aa, Artificial Sequence
  Description of Artificial Sequence synthetic
SSTVSFMN

SEQ ID NO: 11 - DNA, 24 bp, Artificial Sequence
  CDS (1)..(24): Description of Artificial Sequence synthetic construct
agctcaagtgtagcgtatatgaac

SEQ ID NO: 12 - protein, 8 aa, Artificial Sequence
  Description of Artificial Sequence synthetic
SSSVAYMN

SEQ ID NO: 13 - DNA, 24 bp, Artificial Sequence
  CDS (1)..(24): Description of Artificial Sequence synthetic construct
agccagagtgctaagcatatgaac

SEQ ID NO: 14 - protein, 8 aa, Artificial Sequence
  Description of Artificial Sequence synthetic
SQSAKHMN

SEQ ID NO: 15 - DNA, 24 bp, Mus musculus
  CDS (1)..(24)
gccacatccaatttggcttctgga

SEQ ID NO: 16 - protein, 8 aa, Mus musculus
ATSNLASG

SEQ ID NO: 17 - DNA, 24 bp, Artificial Sequence
  CDS (1)..(24): Description of Artificial Sequence synthetic construct
gccacagagaagttggcttctgga

SEQ ID NO: 18 - protein, 8 aa, Artificial Sequence
  Description of Artificial Sequence synthetic
ATEKLASG

SEQ ID NO: 19 - DNA, 24 bp, Artificial Sequence
  CDS (1)..(24): Description of Artificial Sequence synthetic construct
gccacagttaatttggcttctgga

SEQ ID NO: 20 - protein, 8 aa, Artificial Sequence
  Description of Artificial Sequence synthetic
ATVNLASG

SEQ ID NO: 21 - DNA, 24 bp, Artificial Sequence
  CDS (1)..(24): Description of Artificial Sequence synthetic construct
gccacagtgaatttggcttctgga

SEQ ID NO: 22 - protein, 8 aa, Artificial Sequence
  Description of Artificial Sequence synthetic
ATVNLASG

SEQ ID NO: 23 - DNA, 24 bp, Artificial Sequence
  CDS (1)..(24): Description of Artificial Sequence synthetic construct
gccacatccagggcggcttctgga

SEQ ID NO: 24 - protein, 8 aa, Artificial Sequence
  Description of Artificial Sequence synthetic
ATSRAASG

SEQ ID NO: 25 - DNA, 24 bp, Artificial Sequence
  CDS (1)..(24): Description of Artificial Sequence synthetic construct
gccacacagaatttggcttctgga

SEQ ID NO: 26 - protein, 8 aa, Artificial Sequence
  Description of Artificial Sequence synthetic
ATQNLASG

SEQ ID NO: 27 - DNA, 24 bp, Artificial Sequence
  CDS (1)..(24): Description of Artificial Sequence synthetic construct
gccacatccaatttggcttctgga

SEQ ID NO: 28 - protein, 8 aa, Artificial Sequence
  Description of Artificial Sequence synthetic
ATSNLASG